(19) United States
(12) Patent Application Publication
(10) Pub. No.: US 2008/0152674 A1
(43) Pub. Date: Jun. 26, 2008

(54) EPILATION COMPOSITIONS HAVING HIGH TEMPERATURE-SENSITIVITY

(76) Inventor: Joseph M. Luizzi, Newtown, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/962,349

(22) Filed: Dec. 21, 2007

Related U.S. Application Data
(60) Provisional application No. 60/871,497, filed on Dec. 22, 2006.

(51) Int. Cl. A61K 9/00; A61K 8/18; A61Q 7/00; A61K 8/72

(52) U.S. Cl. 424/400; 424/70.1; 424/70.11

(57) ABSTRACT

Embodiments relate to a method of epilating a body surface includes applying to the body surface an epilation composition having a first shear storage modulus and a second shear storage modulus. The first shear storage modulus is less than 0.1% of a second storage modulus. The first shear storage modulus is measured at a first temperature and said second shear storage modulus is measured at a second temperature. The first temperature and the second temperature are no more than 10 Celsius degrees apart, and the first temperature and the second temperature are both within the range of 25° C. and 50° C. The method further comprises removing the epilation composition in a manner sufficient to epilate the skin.

### CROSS REFERENCE TO RELATED APPLICATIONS

This application claims priority to Application No. 60/871,497 filed on Dec. 22, 2006, the entire contents of which are incorporated by reference herein.

### FIELD OF THE INVENTION

This invention relates to methods, compositions, and materials useful for epilation, in particular, to epilation compositions having high temperature sensitivity and uses thereof.

### BACKGROUND OF THE INVENTION

For aesthetic or personal reasons, many people find it desirable to remove unwanted hair from various areas of the human body, e.g., the bikini area, or areas of the face such as around the eyebrows or lips. Methods known for removing hair include shaving, chemical treatment, and physical hair removal. Shaving the hair suffers from the inconvenience of frequently needing to repeat the process, e.g., daily. The use of chemical treatments to cleave the hair shaft (i.e., depilatories) suffers from the drawback of using the chemicals themselves, which generally irritate the skin, have unpleasant odors, and are messy.

Physical hair removal, in which hair is pulled from the skin “by the roots” (broadly termed “epilation”) generally overcomes the problems of chemical irritation, smell, and the requirement for frequent treatment, but additional problems are introduced. In order to provide consumer convenience, ready-to-use products (also known as “cold waxes”) are available. While these products include an epilation composition that does not require heating, this class of composition generally exhibits poor hair removal and often suffers from “cold flow” problems, i.e., the epilation composition is fluid enough to flow in the package, prior to use, often resulting in sticky, messy, or even unusable product.

To address the poor hair removal problems of ready to use products, products that include an epilation composition that requires heating (a so-called “hot wax”) prior to use have been proposed. However, to render a conventional hot wax fluid enough to spread across the skin, the hot wax needs to be heated to a high temperature. This heating step generally requires the use of a microwave, often inconveniently located away from where the individual desires to perform the epilation process. Furthermore, the results of the heating are often unpredictable, usually resulting in not enough heating, thereby causing the individual to either use a wax that cannot spread properly or forcing the user to heat the wax again. Another possible unfortunate result is too much heating of the hot wax, resulting in burning of the skin or forcing the user to wait to let the wax cool down. As such, Applicants have identified the need for an epilation composition that overcomes one or more of the above mentioned drawbacks. Furthermore, Applicants have surprisingly discovered that one or more of the above mentioned drawbacks may be overcome by an epilation composition that has particular temperature-dependent rheological characteristics.

### SUMMARY OF INVENTION

Accordingly, according to a first aspect of the invention, a method of epilating a body surface is provided including the steps of applying to the body surface an epilation composition having a first shear storage modulus and a second shear storage modulus, wherein the first shear storage modulus is less than 0.1% of the second storage storage modulus, and wherein said first shear storage modulus is measured at a first temperature and said second shear storage modulus is measured at a second temperature, and wherein said first temperature and said second temperature are no more than 10 Celsius degrees (C.°) apart, and wherein said first temperature and said second temperature are both within the range of 25° C. and 50° C. The method further includes removing the epilation composition in a manner sufficient to epilate the skin.

According to a second aspect of the invention, a method of epilating a body surface is provided including the steps of applying to the body surface an epilation composition having a first shear storage modulus and a second shear storage modulus, wherein the first shear storage modulus is less than 0.01% of the second shear storage modulus, and wherein said first shear storage modulus is measured at a first temperature and said second shear storage modulus is measured at a second temperature, and wherein said first temperature and said second temperature are no more than 20 C.° apart, and wherein said first temperature and said second temperature are both within the range of 25° C. and 50° C. The method further includes removing the epilation composition in a manner sufficient to epilate the skin.

According to a third aspect of the invention, a method of epilating a body surface is provided including the steps of applying to the body surface an epilation composition that includes a semi-crystalline polymer having crystallizable side chains. The side chains may be hydrophobic, and may include more than 10 carbon items. In one embodiment, the epilation composition further includes a thermochromic material, e.g., a thermochromic material that provides a discernible color change between about 25° C. and 50° C.

According to a fourth aspect of the invention a composite material is provided that includes a substrate; a coating formed upon said substrate, wherein said coating includes an epilation composition described in one of the above three paragraphs. The coating may be formed only on select portions of the substrate in order to increase the flexibility of the composite material. In one embodiment, the coating is formed only across a plurality of discrete portions of a surface of the substrate.

According to a fifth aspect of the invention, a method of epilating a body surface is provided including the steps of urging hot air or hot water across an epilation composition; applying an epilation composition to the skin; and removing the epilation composition in a manner sufficient to epilate the skin. In one embodiment, hot air, such as from a hair drier, is urged across the epilation composition for a time period sufficient to reduce the shear storage modulus to a final shear storage modulus that is less than about 0.1% of an initial shear storage modulus.

According to sixth aspect of the invention, an epilation composition is provided the composition having a first shear storage modulus and a second shear storage modulus, wherein the first shear storage modulus is less than 0.01% of the second shear storage modulus, and wherein said first shear storage modulus is measured at a first temperature and said second shear storage modulus is measured at a second temperature, and wherein said first temperature and said second temperature are no more than 20 C.° apart, and wherein said first temperature and said second temperature are both within the range of 25° C. and 50° C.

### DETAILED DESCRIPTION OF THE INVENTION

The present invention provides compositions, methods, and materials useful for epilation. In particular, the compositions, methods, and materials of the present invention provide the combination of excellent hair removal as well as an ease of use lacking in the prior art.

By “epilation” it is meant the physical and typically forceful removal of the entire hair shaft hair from the body. Epilation includes what is commonly referred to as “waxing;” e.g., applying an epilation composition that bonds to the hair, then applying sufficient force to extract the hair from the body. Epilation compositions may include, for example waxes, polymers, resins, rosin or other bonding materials, optionally compounded with oils, fillers, dyes, and the like. Conventional epilation compositions may be heated, even melted, to provide sufficient tack for hair removal.

According to embodiments of the present invention, a body surface undergoes epilation. By “body surface” it is meant that portion of the body encompassing a surface of the body from which unwanted hairs protrude (i.e., skin) and/or the hairs protruding therefrom. Examples of body surfaces include the bikini area, legs, arms and/or areas of the face such as around the eyebrows or lips.

Note that the term “body temperature” as used herein refers to about 37° C. (98.6° F.). Similarly the term “room temperature” as used herein refers to about 20° C. (68° F.).

### Epilation Composition

Applicants have recognized that conventional epilation compositions and methods fail to provide the highly desirable combination of excellent hair removal together with ease of use. In particular, Applicants note that conventional epilation compositions do not simultaneously possess good fluidity (to spread across and anchor about the hair) and high strength (to promote good hair removal). Nor can conventional compositions readily or quickly switch between a state of high fluidity and a state of high strength. Such a transition for conventional epilation compositions takes place over a broad temperature range and often occurs inhomogeneously in the epilation composition.

The inventors have found that by using an epilation composition that has high temperature sensitivity, the epilation composition can essentially be “switched” between a state that is highly fluid and a state that has high strength. In particular, in one embodiment of the invention, the epilation composition has a first shear storage modulus and a second shear storage modulus. The first shear storage modulus is measured at a first temperature and the second shear storage modulus is measured at a second temperature, the first temperature and said second temperature being no more than 10 C.° apart. The first temperature and the second temperature are also both within the range of 25° C. to 50° C. The ratio of the first shear storage modulus to the second shear storage modulus so determined (hereinafter referred to as the “ten-degree modulus sustain ratio” or “10-MSR”) is less than 0.1%. In a preferred embodiment, the 101-MSR is less than 0.01%. In a further preferred embodiment, the 10′-MSR is less than 0.001%. Compositions of the present invention, by having a low 10′-MSR, have a modulus that falls dramatically with temperature, and therefore have the desirable property of high temperature sensitivity. Measurement of shear storage modulus, G′, is set forth in detail herein.

In another embodiment of the invention, the epilation composition has a first shear storage modulus and a second shear storage modulus. The first shear storage modulus is measured at a first temperature and the second shear storage modulus is measured at a second temperature, the first temperature and said second temperature being no more than 20 C.° apart. The first temperature and the second temperature are also both within the range of 25° C. to 50° C. The ratio of the first shear storage modulus to the second shear storage modulus so determined (hereinafter referred to as the “twenty-degree modulus sustain ratio” or “20′-MSR”) is less than 0.01%. In a preferred embodiment, the 10-MSR is less than 0.001%. In a further preferred embodiment, the 10′-MSR is less than 0.0001%. A low 20°-MSR indicates the desirable property of high temperature sensitivity across a broad temperature range.

Applicants have also found that epilation compositions of the present invention, in certain embodiments, have a latent heat of melting that is at least about 20 J/g C.°. Measurement of latent heat of melting, ΔHₘ, is set forth in detail herein.

### Semi-Crystalline Polymer

In another aspect of the invention, the inventors have found that preferred epilation compositions having high temperature sensitivity include a semi-crystalline polymer. Particularly suitable semi-crystalline polymers are those having crystallizable side chains, of which the chemistry and methods of making are exemplified in U.S. Pat. No. 5,156,911 and U.S. Pat. No. 5,387,450 to Stewart et al., both of which are incorporated by reference. Such polymers generally include a backbone and further include side chains that are capable of entering into a crystalline state. Preferred semi-crystalline polymers are those having crystallizable side chains, include polymers of one or more monomers such as substituted and unsubstituted acrylates, fluoroacrylates, vinyl esters, acrylamides, maleimides, α-olefins, p-alkyl styrenes, alkylvinyl ethers, alkylethylene oxides, triglycerides (e.g. tristearin and pentaerythritol tetrastearate), alkyl phosphazenes and amino acids; polyisocyanates; polyurethanes; and polysiloxanes. Particularly suitable examples of side chains include those having hydrophobic groups, such as those having at least 10 carbon atoms, e.g., linear aliphatic chains such as of C14-C22 acrylates or methacrylates, acrylamides, methacrylamides, and the like.

The semi-crystalline polymer having crystallizable side chains is desirably selected so as to provide temperature sensitivity of shear storage modulus within the temperature range 25° C. and 50° C. that meets the requirements described previously.

The semi-crystalline polymer having crystallizable side chains is also desirably selected so that the epilation compositions are not aggressively tacky at body temperature, and, in some preferred embodiments, non-tacky at body temperature. As such, the epilation composition can be applied at a temperature that is above body temperature; when the epilation composition cools to body temperature, tack is reduced, as is the likelihood of generating residues that are tacky and difficult to remove from the skin or hair.

One suitable example of a semi-crystalline polymer having crystallizable side chains is a poly C10-C30 alkyl acrylate with a molecular weight of about 145,000 and, in its pure state, a (“peak” or “onset”) melting point of 49° C., commercially available as INTELIMER IPA 13-1 from Landec Corporation of Menlo Park, Calif.

While the above mentioned polystearyl acrylate with a molecular weight of about 145,000 is suitable, the inventors have also contemplated the use of higher molecular weight semi-crystalline polymers having crystallizable side chains, and, in particular, higher molecular weight semi-crystalline polymers having crystallizable side-chains. For example, an acrylic semi-crystalline polymer having crystallizable side chains and having a molecular weight from about 140,000 to about 500,000 may be desirable to resist flow at elevated temperature (has reduced runniness) moreso than lower molecular weight polymers. This attribute may be suitable to keep the epilation composition to stay in place when heated.

The semi-crystalline polymer may be used in any concentration that is suitable to provide the desired temperature sensitivity of shear storage modulus. In one embodiment of the invention, the concentration (all concentrations in this specification are by weight unless specifically indicated otherwise) of semi-crystalline polymer is from about 2% to about 100%, preferably from about 3% to about 50%, more preferably from about 15% to about 40%, most preferably from about 20% to about 40%.

### Other Components of the Epilation Composition

The epilation composition may include components other than the semi-crystalline polymer. In one embodiment the epilation composition includes a diluent to aid in delivering the composition across an expanse of skin that is to be epilated. The diluent is generally selected to be non-irritating to the skin and readily dissolves, disperses or suspends other ingredients in the composition. In preferred embodiments of the invention, the epilation composition has a high concentration or predominance of hydrophobic ingredients. In this embodiment, the diluent is preferably selected so as to be hydrophobic. Conversely, for compositions in which there is a high concentration or predominance of hydrophilic ingredients, the diluent may be selected so as to be hydrophilic. The diluent may be volatile, moderately volatile, or non-volatile at room temperature. The diluent may be selected from a liquid diluent, a solid diluent, or combinations thereof.

The liquid diluent is a liquid at room temperature. In one embodiment of the invention the liquid diluent is a hydrophobic liquid. Suitable examples include mineral oils; silicone oils; vegetable oils; or other volatile or non-volatile hydrophobic liquids such as liquid turpenes and the like. Other liquid hydrophobic diluents that have some polarity may also be suitable. Suitable examples of such include esters, such as esters of fatty acids (e.g., triglycerides) aldehydes, ketones, phenols. In another embodiment the liquid diluent is a hydrophilic liquid at room temperature such as water, propylene glycol, water-miscible alcohols. In one embodiment, the epilatory composition is substantially free (i.e., having less than about 5%, preferably less than about 1%) of hydrophilic liquids such as water.

The liquid diluent may be present in a concentration that does not interfere with the ability of the composition to provide tack in use. In addition, the liquid diluent is desirably present in a concentration that does not interfere with the ability of the composition to achieve one or more of the previously mentioned desirable ranges for modulus sustain ratio or ΔHₘ. Furthermore, in certain embodiments it is desirable not to include such a high percentage of liquid diluent that the composition is particularly tacky at room temperature. In one embodiment of the invention, the liquid diluent is hydrophobic and is present in a concentration that is from about 0% to about 15%.

The solid diluent is a solid, paste, gel, or the like at room temperature and is generally a non-polymerized or moderately polymerized material. Particularly notable solid diluents are hydrophobic solid diluents such as fats, i.e., glyceryl esters of fatty acids and waxes, i.e., mixtures of esters, not necessarily esters of glycerol, often including long chain alkyl groups; or mineral waxes such as paraffin wax or petrolatum; and silicone waxes (derived from silicon, oxygen, oxygen, and hydrogen) such as alkylmethyl-dimethylsiloxanes. The solid diluent may melt over a temperature range that is within from about 30° C. to about 75° C., preferably from about 35° C. to about 65° C. One particularly notable solid diluent is a silicone wax that melts over a temperature range that is from about 36° C. to about 56° C., and is commercially available as a mixture of stearoxytrimethylsilane and stearyl alcohol, DC 580 Wax from Dow Corning of Midland, Mich. Another particularly suitable solid diluent is petrolatum. Note that while hydrophobic solid diluents are particularly notable, a hydrophilic solid diluent such as hydrophilic film-forming polymer (e.g., polysaccharides such as starches, cellulose polymers, gums, and the like) is also contemplated, particularly for embodiments in which the epilation composition is aqueous, i.e., has water as a continuous phase.

The solid diluent may be present in a concentration that does not interfere with the ability of the composition to provide tack in use. In addition, the solid diluent is desirably present in a concentration that does not interfere with the ability of the composition to achieve one or more of the previously mentioned desirable ranges for modulus sustain ratio or ΔHₘ. In one embodiment of the invention, the concentration of solid diluent, preferably a hydrophobic solid diluent, more preferably a wax, is from about 10% to about 50%, preferably from about 15% to about 35%, such as from about 20% to about 30%.

In one embodiment the epilation composition includes a tackifying resin to help the epilation composition adhere to the hair to be epilated, as well optionally to provide some melt stiffness and resistance to fluidity at elevated temperature. The tackifying resin desirably is phase compatible with other ingredients in the epilation composition and has a melting point greater than about 55° C., preferably from about 65° C. to about 100° C., more preferably from about 60° C. to about 85° C. Furthermore, the tackifying resin preferably has a weight average molecular weight that is greater than about 300, preferably from about 400 to about 1000.

Examples of suitable tackifying resins include hydrophobic tackifying resins such as hydrocarbon resins such as rosins, modified rosins and other terpene resins; cyclic aliphatic resins; other tackifying resins known in the art, as well as certain high melting point “microcrystalline waxes”. One particularly suitable hydrophobic tackifying resin is a heat stable styrenated terpene resin, commercially available as ZONATAC M106, commercially available from Arizona Chemical of Jacksonville, Fla.

Note that while hydrophobic solid diluents and hydrophobic tackifying resins are particularly notable for use in the inventive epilation compositions, hydrophilic tackifying materials such as polysaccharides including one or more sugars (e.g., sucrose) is also contemplated, particularly for embodiments in which the epilation composition is aqueous.

The concentration of tackifying resin may be high enough to provide adhesion to the hair, particularly when heated. However, it may be desirable to keep the concentration of tackifying resin low enough such that body temperature tack is not high enough so that residue on the skin is increased. In one embodiment the concentration of tackifying resin is from about 10% to about 70%, such as from about 30% to about 60%.

In another embodiment of the invention, the epilation composition includes a temperature-indicating material that is designed to provide a visual, olfactory, or auditory sensation that varies with temperature. In one embodiment, the temperature-indicating material is a thermochromic material, i.e., a material that changes color or visual appearance upon being heated. It is further desirable that the theremochromic material return to its original appearance upon being cooled, i.e., the visual appearance change is reversible.

In one notable embodiment, the thermochromic material is designed to undergo a discernible color change when subject to a temperature transition that occurs between body temperature and about 50° C., such as between about 40° C. and about 50° C. The discernible color change may coincide with the range over which the epilation composition shows a most dramatic change in shear storage modulus.

Suitable thermochromic materials include inks based on thermochromic chemistries, such as those utilizing cholesteric, ester or acrylic liquid crystal phase change materials. Commercial examples of these include ThermaSOLV W/B Non-woven Inks from AIC/Sun Chemical (Saint Aignan de Grand Lieu, France) and Waterbased Flexo Inks from Chromatic Technologies, Inc. (Colorado Springs, Colo.).

The epilation composition of the present invention may be particularly suitable for including ingredients that are not included in conventional “hot wax” epilation compositions because such ingredients are prone to degradation when conventional epilation material are heated to high temperatures. As such, the epilation composition may include ingredients that provide beneficial biological/biochemical activity to the subject, such as those that may be suitable for retarding hair growth (e.g., extracts of soy for example as disclosed in U.S. Pat. No. 6,55,143, U.S. Patent Application Publication Nos. US 2002/0160062, US 2003/00604048, US 2005/0126353 the disclosures of which are hereby incorporated by reference) and U.S. application Ser. No. 10/253,236 filed Sep. 23, 2002, chemically cleaving hair shafts (e.g., thioglycolates), anti-inflammatory agents e.g., benoxaprofen, centella asiatica, bisabolol, extracts of feverfew, green tea extract, green tea concentrate, hydrogen peroxide, salicylates, oat oil, chamomile, dipotassium glycyrrhizate), anti-edema agents (e.g., bisabolol natural, synthetic bisabolol, corticosteroids, beta-glucans), analgesics (e.g., for example, “caine” molecules such as benzocaine, dibucaine, lidocaine; benzyl alcohol, camphor, juniper tar, menthol, methyl nicotinate, methyl salicylate, phenol, resorcinol), counterirritants (e.g., allantoin, camphor, menthol, methyl salicylate, peppermint and clove oils, ichtammol).

The epilation composition may also include particulate materials dispersed therein to provide desirable texture, rheology, or reduced material cost. Suitable particulate materials include inorganic particulates such as various refined minerals, oxides, silicates, and the like. Examples include silicon oxides, aluminum oxides, zinc oxides, titanium oxides, boron nitrides, talc, gypsum, calcite among others, and combinations thereof. Other suitable particulate materials include organic particulates such as synthetic polymers that may be chemically crosslinked such as silicone elastomers, natural particulate materials including proteins or polysaccharides derived from vegetable or animal sources. The particulate material may be coated such as with silanol or hydrophobic moieties in order to enhance compatibility with the remainder of the epilation formulations. The particulate material may be present in any suitable concentration, such as from about 0% to about 10%.

The epilation composition may include other functional components such as dyes, fragrances, essential oils, pH adjusters (for aqueous compositions), dispersants, emulsifiers, wetting agents, rheology modifiers, preservatives, sequestering agents, and antioxidants.

Epilation compositions of the present invention may be applied to a body surface in various manners. In one embodiment of the invention the epilation composition is formed on a substrate or carrier. The substrate is designed to provide convenient manufacturing, packaging, transportation, and/or use of the epilation composition. As such, the substrate may have one or more desirable features, including: water-resistance or water-insolubility to prevent the substrate from degrading prior to use; small size, low weight or thickness for convenience; flexibility such that the substrate is readily flexed across curved areas of the body; readily wettable with the epilatory composition for ease of coating; thermally resistant to prevent thermal degradation of the substrate for the situation in which the epilation composition is heated prior to coating on the substrate. The substrate may include fibrous materials such as paper, cellophane or non woven fibers; thermoplastic films, and combinations thereof. The substrate may include a surface that has been rendered hydrophobic.

The epilation composition may be coated onto the substrate in an amount suitable to promote good epilation. For example, in one embodiment of the invention, the epilation composition may be coated to from about 50 grams per square meter (gsm) to about 400 gsm.

The epilation composition may be formed on the substrate by coating techniques such as slot coating, roll coating, spraying, and the like. In one desirable embodiment, the epilation composition is provided between two substrates, such that when the two substrates are pulled apart, epilation composition remains behind on both substrates so that each may be used for epilation.

Although the epilation composition may be formed on or between the substrate in a continuous manner (i.e, only one continuous region of epilation composition exists on or between the substrates), this is not required. In one embodiment, the epilation composition is formed only on select portions of the substrate. In one embodiment, the coating is formed only across a plurality of discrete portions of a surface (e.g., a body contactable surface) of the substrate.

The discrete portions or “islands” may be of any shape including, but not limited to, lines, waves, interconnected patterns, circular dots, hexagons, hearts, diamonds, flowers, butterflies, rectangles, stars, triangles and the like. These islands may individually have an area capable of contacting the skin that is from about 3 mm² to about 300 mm², such as from about 10 mm² to about 150 mm², such as from about 25 mm² to about 100 mm². The percentage of substrate area that is covered by epilation composition to the substrate that is absent expiation composition may be from about 20% to about 95%, preferably from about 30% to about 80%, and most preferably from about 40% to about 70%. By having a percent coverage of epilation composition that is in the specified ranges, it is possible to provide sufficient delivery of epilation composition to the skin to be treated, yet also increase the flexibility of the substrate, such that it readily flexes around curved regions of the body. This flexibility is possible even if the epilation composition itself is somewhat stiff, such as may be the case before heat is applied.

In another embodiment of the invention, the substrate has a thermochromic material formed thereon such that the thermochromic material defines an image that can be seen by a user. The thermochromic material may be similar to the thermochromic material described above useful in the composition. The thermochromic material may be applied to the substrate via any suitable means (e.g., flexographic, gravure, or ink jet printing) and with any suitable binders or resins that provides compatibility with the thermochromic material and good adhesion to the substrate and good image formation.

Epilation compositions of the present invention may be used in various manners. For embodiments in which the epilation composition is formed on or between substrates, the substrates are optionally warmed by methods described below in order to improve the ability to peel the substrates apart. The side that is coated with epilation composition is applied to skin to be epilated. To improve the flexibility of the coated substrate, the stiffness of the epilation composition may be reduced by raising the temperature thereof. This may be accomplished by applying heat to the coated substrate, either while applied to the skin or before the coated substrate is applied to the skin or when it is in contact with the skin by various means such as (1) heating the coated substrate, such as by urging hot air across the coated substrate (e.g., by using a hair drier) or running under warm water; (2) applying the coated substrate to skin that has been pre-warmed such as via a hot shower, hot bath or hot washcloth; (3) rubbing or holding the coated substrates in one's hands to provide heat thereto. This temperature change may be sufficient to effect a phase change in a semi-crystalline polymer that is present in the epilation composition. For embodiments in which either the epilation composition or the substrate includes a thermochromic material, the heat may be applied for a time period sufficient to effect a change in visual appearance of the thermochromic material (signaling to the user enough heat has been applied). For example, the thermochromic material may have its temperature raised to a final temperature from about 25° C. to about 50° C.

The coated substrate is flexed around curved areas of the skin and contact is provided between the hairs to be removed and the epilation composition. To enhance contact between the epilation composition and the hairs, the substrate may be pressed firmly on to the skin for a period from a few seconds to as long as about one minute. This time period may be sufficient to effect a phase transition in a semi-crystalline polymer present in the epilation composition. For embodiments wherein the epilation composition includes the thermochromic material, the user may wait a period of time for an appearance change in the thermochromic material (indicating sufficient cooling has taken place to promote good hair removal and low likelihood of undesirable residue on the skin). The substrate is then removed in a manner sufficient to epilate the skin, leaving hairs attached to the coating of epilation composition.

Note that if the epilation composition and/or substrate has been inadvertently applied to a portion of the body that is not ideal, unlike conventional epilation compositions, epilation compositions of the present invention are readily repositioned to a more ideal location on the body. In some cases this can be accomplished by applying a little burst of heat such as from a hair drier to render the composition more fluid.

While the above use has been described with reference to a substrate, the epilation composition need not be pre-formed on the substrate. Alternatively, the epilation composition may be applied to the skin via a device (e.g., a spatula) such as after independently heating the epilation composition and its container in a water bath, microwave, etc. This may be performed until the optional thermochromic material changes appearance. The epilation composition is then spread across the skin, an uncoated substrate is placed into contact with the epilation composition that was previously applied to the skin, the epilation composition is allowed to cool, and the substrate is removed in a manner to epilate the skin.

### Procedure for Measuring Shear Storage Modulus of the Epilation Composition

Shear storage modulus is determined using the method described below: a sample of the composition to be tested is placed in a parallel plate rheometer, e.g., an Ares 2000 Solids Analyzer from TA Instruments of Wilmington Del. The sample is tested in parallel plate geometry using 25 mm diameter, approximately, 2 mm thickness in an oscillatory shear, using a shear rate of 10 radians per seconds. The samples are subjected to continuous oscillatory shear as the temperature is increased from a starting temperature of 20° C. to an ending temperature of 100° C. Measurements are taken at increments of 2-3 Celsius degrees (C.°). This type of rheology measurement, conducted at a fixed shear rate while varying temperature is known as a “temperature sweep.”

In order to characterize the maximum amount of temperature sensitivity of a particular epilation composition, from the temperature sweep, one may determine the minimum 10′-MSR and the minimum 20′-MSR (defined previously, these parameters are essentially the greatest amount G′ collapses over a 10 degree temperature range and a 20 degree temperature range, respectively). Compositions may be compared by examining the minimum 10°-MSR and the minimum 20′-MSR. A composition having a low ratio indicates the modulus falls dramatically with temperature and has the desirable property of high temperature sensitivity. The inventive epilation compositions may have one or more of a 101-MSR less than 0.1% and a 20′-MSR less than 0.01%.

### Procedure for Measuring Latent Heat of Melting of the Epilation Composition

Latent heat of melting, ΔHₘ is determined using a Perkin-Elmer Differential Scanning Calorimeter (Wellesley, Mass.). A sample having a mass of about 5.0 mg is heated from 25° C. to about 80° C., cooled to about 25° C., and re-heated to about 80° C. The heating and cooling are conducted at a temperature ramp rate of 10 C.°/min.

The advantages of the invention and specific embodiments of the skin care compositions prepared in accordance with the present invention are illustrated by the following examples.

### EXAMPLES OF INVENTIVE EPILATION COMPOSITIONS AND METHODS

Specific examples of inventive epilation compositions and methods are described below. Comparative examples are also provided.

### Inventive Examples 1-4

The components shown in Table 1 were used to make inventive epilation compositions.

TABLE 1

| Trade Name | Chemical Name | Primary Function |
|---|---|---|
| INTELIMER IPA 13-1 | Poly C10-C30 alkyl | Semi-crystalline |
| | acrylate | polymer |
| ZONATAC M106 | Styrenated terpene resin | Tackifying Resin |
| DC 580 Wax | Stearoxytrimethylsilane | Solid Diluent |
| | and stearyl alcohol | |
| Petrolatum | Petrolatum | Solid Diluent |

TABLE 2

| | Weight Percent of Ingredient | | | |
|---|---|---|---|---|
| | INTELIMER | | | |
| Ref. | 13-1 | ZONATAC M106 | DC 580 Wax | Petrolatum |
| Ex A | 40 | 45 | 15 | 0 |
| Ex B | 20.625 | 35.9375 | 33.125 | 10.3125 |
| Ex C | 33.125 | 35.9375 | 28.125 | 2.8125 |

Table 2 above shows the concentrations of the various ingredients used to make the inventive epilation compositions.

Table 3 below is a summary table that shows the of the temperature sensitivity of inventive epilation compositions. Temperatures, T₁ and T₂ were chosen to provide the minimum value for 10′-MSR. Raw rheology data is provided in Appendix 1.

The inventive epilation compositions above were prepared by blending the ingredients at about 70° C., placing them in aluminum pans and allowing them to cool. Samples were melted and laminated to cotton strips having dimensions of about 30 mm×100 mm, with a basis weight of 200 gsm. The strips were allowed to cool to room temperature and were then placed a skin surface to be epilated. The strips were heated with a hand-held hair drier water until the composition was observed to have “melted.” The strips were then allowed to cool about 1-2 minutes and were rapidly peeled away from the skin. Excellent hair removal was observed.

Examples A, B and C were also tested for latent heat of melting, ΔHₘ. ΔHₘ was determined to be 49.6 J/gram C.°, 62.7 J/gram C.°, and 65.5 J/gram C.° respectively.

TABLE 3

| | T₁ | T₂ | ΔT | G′₁ | G′₂ | |
|---|---|---|---|---|---|---|
| Ref. | (° C.) | (° C.) | (° C.) | (dynes/cm²) | (dynes/cm²) | 10°-MSR (%) |
| Ex A | 31.51 | 39.77 | 8.26 | 3.07 × 10⁷ | 202.8 | 0.00066 |
| Ex B | 34.93 | 44.69 | 9.76 | 3.17 × 10⁶ | 0.606 | 0.000019 |
| Ex C | 33.87 | 43.85 | 9.98 | 1.01 × 10⁷ | 5.53 | 0.00005 |

Table 4 below is a summary that shows the temperature sensitivity of the inventive epilation compositions. Temperatures, T₁ and T₂ were chosen to provide the minimum value for 20′-MSR. Raw rheology data is provided in Appendix 1.

TABLE 4

| | T₁ | T₂ | ΔT | G′₁ | G′₂ | |
|---|---|---|---|---|---|---|
| Ref. | (° C.) | (° C.) | (° C.) | (dynes/cm²) | (dynes/cm²) | 20°-MSR (%) |
| Ex A | 26.26 | 45.81 | 19.55 | 7.11 × 10⁷ | 26.60 | 0.000037 |
| Ex B | 32.93 | 44.69 | 11.76 | 4.48 × 10⁶ | 0.606 | 0.000014 |
| Ex C | 27.86 | 47.77 | 19.91 | 3.86 × 10⁷ | 1.09 | 0.0000028 |

### Comparative Examples 1-4

The following prior art epilation compositions (Comparative examples 1-4) were evaluated:
Comparative Example 1, Surgiwax Brazilian Hair Remover.
Comparative Example 2, Nair Microwave Wax, commercially available from Church & Dwight, Co. Inc., of Princeton, N.J.
Comparative Example 3, Veet Warmwachstiegel, commercially available from Reckitt Benckiser, PLC of Berkshire, United Kingdom
Comparative Example 4, Sally Hansen Spa Wax Microwave Formula, commercially available from Sally Hansen of Uniondale, N.Y.

Table 5 is a summary table that shows the of the temperature sensitivity of the comparative epilation compositions. Temperatures, T₁ and T₂ were chosen to provide the minimum value for 10′-MSR. Raw rheology data is provided in Appendix 1.

Comparative Examples 1, 2, 3 and 4 were also tested for latent heat of melting, ΔHₘ. ΔHₘ was determined to be 17.9 J/gram C.° for Comparative Example 1. Comparative Examples 2-4 did not exhibit any detectable phase transition and thus, no ΔHₘ is reported.

TABLE 5

| | | | | | | 10°-MSR |
|---|---|---|---|---|---|---|
| Ref. | T₁ | T₂ | ΔT | G′₁ | G′₂ | (%) |
| Comp 1 | 38.2 | 48.2 | 10.0 | 4.83 × 10⁶ | 1.24 × 10⁶ | 25.67 |
| Comp 2 | 28.78 | 38.59 | 9.81 | 1.84 × 10⁵ | 322.8 | 0.175 |
| Comp 3 | 34.89 | 44.63 | 9.74 | 7837 | 120.4 | 1.54 |
| Comp 4 | 27.91 | 36.86 | 9.95 | 1589 | 89.3 | 5.62 |

Table 6 is a summary table that shows the of the temperature sensitivity of comparative epilation compositions. Temperatures, T₁ and T₂ were chosen to provide the minimum value for 20′-MSR. Raw rheology data is provided in Appendix 1.

TABLE 6

| | | | | | | 20°-MSR |
|---|---|---|---|---|---|---|
| Ref. | T₁ | T₂ | ΔT | G′₁ | G′₂ | (%) |
| Comp 1 | 29.18 | 48.2 | 19.02 | 6.14 × 10⁶ | 1.24 × 10⁶ | 20.2 |
| Comp 2 | 29.78 | 49.6 | 19.82 | 1.56 × 10⁵ | 30.68 | 0.0197 |
| Comp 3 | 25.9 | 44.63 | 19.73 | 1.36 × 10⁵ | 120.4 | 0.09 |
| Comp 4 | 25.9 | 44.64 | 19.74 | 1955 | 32.43 | 1.66 |

The preceding data indicates the temperature sensitivity of the inventive examples is excellent. Considering first the 10°-MSR calculations (Tables 3 and 5): Inventive Example A, G′ falls to 0.00066% of its original value over a temperature change that is only 8.26 C.°. The temperature sensitivity of the comparative examples is poor by comparison. Even for the most temperature sensitive, Comp. 2, G′ falls only to 0.175% of its original value. This is about 1000 times less temperature sensitive than the inventive examples.

Similarly, considering the 20′-MSR calculations (Tables 4 and 6): the inventive examples have extremely low 20′-MSR values. Inventive Example C, G′ falls to 0.0000028% of its original value over a temperature change that is only 19.91 C.°. Even the most temperature sensitive Comp. 2, G′ falls only to 0.0197% of its original value. This is again about 1000 times less temperature sensitive than the inventive examples.

I claim:

1. A method of epilating a body surface, comprising:
applying to the body surface an epilation composition having a first shear storage modulus and a second shear storage modulus, wherein the first shear storage modulus is less than 0.1% of the second storage storage modulus, and wherein said first shear storage modulus is measured at a first temperature and said second shear storage modulus is measured at a second temperature, and wherein said first temperature and said second temperature are no more than 10 Celsius degrees (C.°) apart, and wherein said first temperature and said second temperature are both within the range of 25° C. and 50° C.; and
removing the epilation composition in a manner sufficient to epilate the skin.

2. The method of claim 1, wherein said first shear storage modulus is less than 0.01% of the second shear storage modulus.

3. The method of claim 1, wherein said second temperature are no more than 20 C.° apart, and wherein said first temperature and said second temperature are both within the range of 25° C. and 50° C.

4. The method of claim 2, wherein said second temperature are no more than 20 C.° apart, and wherein said first temperature and said second temperature are both within the range of 25° C. and 50° C.

5. The method of claim 1, wherein said epilation composition comprises a semi-crystalline polymer having crystallizable side chains.

6. The method of claim 1, wherein said side chains comprise include more than 10 carbon items.

7. The method of claim 1, wherein said epilation composition comprises a thermochromic material that provides a discernible color change between about 25° C. and 50° C.

8. A composite material, comprising:
a substrate; and
a coating formed upon said substrate, wherein said coating includes an epilation composition of claim 1.

9. The composite material of claim 1, wherein the epilation composition comprises a semi-crystalline polymer having crystallizable side chains.

10. The composite material of claim 1, wherein said coating is formed only across a plurality of discrete portions of a surface of the substrate.

11. A method of epilating a body surface, comprising:
urging hot air or hot water across an epilation composition; applying an epilation composition to the skin; and
removing the epilation composition in a manner sufficient to epilate the skin.

12. The method of claim 11, wherein hot air is urged across said epilation composition.

13. The method of claim 11, wherein hot air is urged across said epilation composition for a time period sufficient to reduce the shear storage modulus to a final shear storage modulus that is less than about 0.1% of an initial shear storage modulus.

14. The method of claim 11, wherein the epilation composition is the epilation composition of claim 1.
